(19) United States
(12) Patent Application Publication
(10) Pub. No.: US 2017/0100575 A1
(43) Pub. Date: Apr. 13, 2017

(54) Pre-filled Pen Type Applicator with Novel Topical Acne Preparations

(71) Applicant: CLIXIT, LLC, Orlando, FL (US)

(72) Inventors: Karen Poirier, Orlando, FL (US); Diane Heinz, Longwood, FL (US)

(73) Assignee: CLIXIT, LLC, Orlando, FL (US)

(21) Appl. No.: 15/268,701

(22) Filed: Sep. 19, 2016

Related U.S. Application Data
(60) Provisional application No. 62/239,187, filed on Oct. 8, 2015.

(51) Int. Cl. A61M 35/00; A61K 47/32; A61K 47/18; A61K 47/10; A61K 47/46; A61K 31/192; A61K 33/04; A61K 47/02; A61K 47/12; A61K 47/34; A61K 47/08; A61K 47/22

(52) U.S. Cl. CPC A61M 35/003; A61K 47/08; A61K 47/32; A61K 47/183; A61K 47/10; A61K 47/22; A61K 31/192; A61K 47/18; A61K 33/04; A61K 47/02; A61K 47/12; A61K 47/34; A61K 47/46

(57) ABSTRACT

The present invention relates to a pen-type, pressurized cosmetic applicator with a tubular applicator tip. The invention is filled with a novel, topical acne preparation

### CROSS-REFERENCE TO RELATED APPLICATIONS

This application makes reference to U.S. Prov. App. No. 62/239187 filed Oct. 8, 2015 and incorporates it in its entirety by reference.

### FIELD OF THE INVENTION

The present invention relates to a pen-type cosmetic applicator generally in the form shown in FIG. 1 with an applicator tip generally in the form of FIG. 2. The invention is filled with a novel, topical acne preparation.

### BACKGROUND OF THE INVENTION

Acne is a pervasive condition suffered by millions of teenagers and adults. Generally associated with less than fastidious cleaning regimens and/or hyperactive dermal glands, acne is suffered by virtually all human beings. Unsurprisingly, acne treatments are virtually universal. Whether applied via a finger, a pad, a spray bottle, a tube, or numerous other techniques, acne treatments are ubiquitous.

Nevertheless, the prior art is devoid of a portable pen-shaped instrument for transporting and applying acne medication. Given the ubiquity of the condition, a pen shaped applicator containing acne medication would be useful.

### SUMMARY OF THE INVENTION

An exemplary embodiment of the invention comprises a hollow cylindrical reservoir filled with preparation. Affixed at one end of the reservoir is a pressurizing button. Affixed at the other end of the device is a depository tip. The depository tip is protected by a cover when the device is not in use.

The reservoir is comprised of plastic or aluminum. The pressurizing button is also composed of plastic or aluminum and is mated to a pressurizing module. The pressurizing module is mounted inside the reservoir. The balance of the reservoir is filled with preparation. Affixed to the other end of the reservoir is the depository tip. The depository tip is protected by a cover when the device is not in use. Both the depository tip and the cover are comprised of plastic or aluminum. The depository tip may be comprised of other materials such as steel or ceramic.

An exemplary embodiment of the topical acne preparation is comprised of: camphor, carbomer, deionized water, EDTA, ethanol 40b, ethylhexylglycerin, penoxyethanol, propylene carbonate, salicylic acid, and triethanolamine.

An alternative embodiment of the topical acne preparation is comprised of: sulfur, camphor, carbomer, deionized water, EDTA, triethanolamine, ethanol alcohol, propylene carbonate, salicylic acid, and phenoxvethanol.

An exemplary embodiment of the invention is used in the following manner: First, the user removes the cover from the depository tip. Next, the user places the depository tip where acne medication is desired on the skin. Next, the user actuates the pressurizing button (and affixed pressurizing module) with a finger press. Next, the pressurizing module pressurizes the preparation in the reservoir. Next, pressurized preparation is forced through the depository tip. Next, preparation is deposited on the skin where desired. Next, the user manually works the preparation into the skin with a finger or a similar device. Next, the user replaces the cover over the depository tip.

### BRIEF DESCRIPTION OF THE DRAWINGS

FIG. 1 is a perspective view of the blemish eraser with the cap over the depository tip.

FIG. 2 is a perspective view of the blemish eraser with the cap removed from the depository tip.

### DETAILED DESCRIPTION OF THE PREFERRED EMBODIMENT

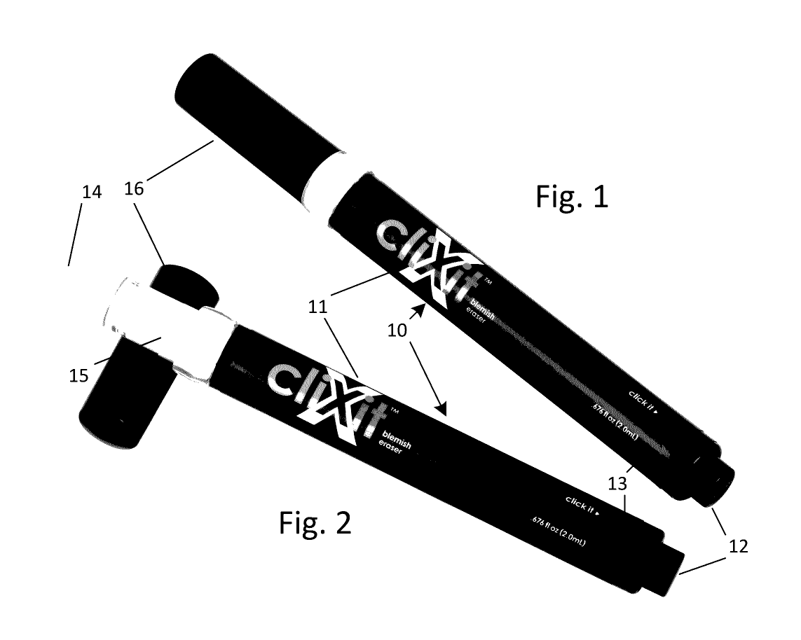

Referring now to FIG. 1 and FIG. 2, blemish eraser 10 is shown. Blemish eraser 10 is comprised of reservoir 11. Reservoir 11 is an elongated hollow shaft. Affixed to one end of reservoir 11 is pressurizing button 12. Pressurizing button 12 is, in turn, coupled to pressurizing module 13 inside reservoir 11. Affixed to the other end of reservoir 11 is depository tip 14. Depository tip 14 is shaped to possess a mating flange 15. When blemish eraser 10 is not in use, cap 16 is placed over depository tip 14 such that the inner surface of cap 16 is intimately associated with mating flange 15.

Blemish eraser 10 is constructed of plastic or aluminum or similar such substances. Multi-material construction is readily conceivable. For example reservoir 11 may be constructed of aluminum while the balance of blemish eraser 10 is constructed of plastic.

Pressurizing module 13 inside reservoir 11 is constructed such that each push of pressurizing button 12 causes an equivalent, additive decrease in the amount of space inside reservoir 11 in which preparation may exist. This causes the preparation inside reservoir 11 to become pressurized and to be expelled though depository tip 14.

An exemplary embodiment of reservoir 11 is filled with a preparation comprised of: camphor, carbomer, deionized water, EDTA, ethanol 40b, ethylhexylglycerin, penoxyethanol, propylene carbonate, salicylic acid, and triethanolamine.

An alternative embodiment of the topical acne preparation is comprised of: sulfur, camphor, carbomer, deionized water, EDTA, triethanolamine, ethanol alcohol, propylene carbonate, salicylic acid, and phenoxyethanol.

In this alternative embodiment, sulfur is used as an antiseptic. Although the amount of sulfur in the preparation may range from about 7% to about 13%, this alternative embodiment is best formulated with a sulfur concentration of about 10%. Similarly, salicylic acid is added to serve as an antimicrobial agent. Although the amount of salicylic acid added to the preparation may range below about 7%, this alternative embodiment is best formulated with a salicylic acid concentration less than about 5%.

Other preparations may be used to fill reservoir 11. These alternative preparations comprise ingredients selected from the following list: deionized water; isopropyl alcohol; zinc oxide; camphor; propylene glycol; talc; ethylhexylglycerine; phenoxyethanol; propylene carbonate; titanium dioxide; butylene glycol; sodium hydroxide; carbomer; potassium sorbate; allantoin; salicylic acid; dimethicone; kaolin; phyto-plant extract; sulphur; benzoyl peroxide; seaweed extract; tea tree oil; azulene extract; EDTA; ethanol 40b; and triethanolamine.

An exemplary embodiment of the invention is used in the following manner: First, the user removes cap 16 from depository tip 14. Next, the user places depository tip 14 near where the preparation is desired on the skin. Next, the user actuates pressurizing button 12 (and affixed pressurizing module 13) with a finger press. Next, pressurizing module 13 pressurizes the preparation in reservoir 11 by causing an equivalent, additive decrease in the amount of space inside reservoir 11 in which the preparation may exist. Next, pressurized preparation is forced through depository tip 14. Next, preparation is deposited on the skin where desired. Next, the user manually works the preparation into the skin with a finger or a similar device. Next, the user replaces cap 16 over depository tip 14.

1. A blemish eraser comprising:
a. a hollow reservoir;
b. a pressurizing button affixed to a pressurizing module wherein the pressurizing module is mounted inside the hollow reservoir and is capable of pressurizing the contents of the hollow reservoir;
c. a depository tip fluidically coupled to the hollow reservoir; and
d. an acne treatment preparation inside the hollow reservoir.

2. A blemish eraser of claim 1 further comprising a cap capable of covering the depository tip.

3. A blemish eraser of claim 1 wherein the acne treatment preparation is comprised of:
a. camphor;
b. carbomer;
c. deionized water;
d. EDTA;
e. ethanol 40b;
f. ethylhexylglycerin;
g. penoxyethanol;
h. propylene carbonate;
i. salicylic acid; and
j. triethanolamine.

4. A blemish eraser of claim 1 wherein the acne treatment preparation is comprised of:
a. sulphur;
b. camphor;
c. carbomer;
d. deionized water;
e. EDTA;
f. triethanolamine;
g. ethanol alcohol;
h. propylene carbonate;
i. salicylic acid; and
j. phenoxyethanol.

5. A blemish eraser of claim 4 wherein the concentration of sulfur is about 10%.

6. A blemish eraser of claim 4 wherein the concentration of salicylic acid is less than about 5%.

7. A blemish eraser of claim 1 wherein the acne treatment medication is comprised of ingredients selected from the following list:
a. deionized water;
b. isopropyl alcohol;
c. zinc oxide;
d. camphor;
e. propylene glycol;
f. talc;
g. ethylhexylglycerine;
h. phenoxyethanol;
i. propylene carbonate;
j. titanium dioxide;
k. butylene glycol;
l. sodium hydroxide;
m. carbomer;
n. potassium sorbate;
o. allantoin;
p. salicylic acid;
q. dimethicone;
r. kaolin;
s. phyto-plant extract;
t. sulphur;
u. benzoyl peroxide;
v. seaweed extract;
w. tea tree oil;
x. azulene extract;
y. EDTA;
z. ethanol 40b; and
aa. triethanolamine.
